(19) United States
(12) Patent Application Publication
(10) Pub. No.: US 2026/0273123 A1
(43) Pub. Date: Sep. 17, 2026

(54) DEVICE AND METHOD FOR DIFFUSING VOLATILE SUBSTANCES

(71) Applicant: ZOBELE HOLDING SPA, TRENTO (IT)

(72) Inventors: Sergio LUQUE VERA, TRENTO (IT); Jesus Ortando RAMIREZ ALVARADO, BARCELONA (ES); Marc MALGOSA BROTO, BARCELONA (ES)

(21) Appl. No.: 19/168,010

(22) PCT Filed: Mar. 22, 2024
(86) PCT No.: PCT/EP2024/057824

§ 371 Date: Sep. 23, 2025

(30) Foreign Application Priority Data
Mar. 23, 2023 (ES) P202330238

(51) Int. Cl. A61L 9/03; H05B 1/02; H05B 3/06

(52) U.S. Cl. CPC A61L 9/037; H05B 1/0244; H05B 3/06; A61L 2209/135; H05B 2203/007

(57) ABSTRACT

The device for diffusing volatile substances comprises a container (1) containing a liquid with volatile substances, a wick (2) partially housed in the container (1), which is impregnated with the liquid with volatile substances contained in the container (1); and a heater (3), which heats the wick (2) to promote diffusion of the volatile substances, the heater (3) comprising a plurality of heating elements (31) arranged around the wick (2). It provides a device and a method for diffusing volatile substances, whereby the user perceives the fragrance from a short period of time after switching on the device, due to the low thermal inertia of the heater.

### OBJECT OF THE INVENTION

The present invention relates to a device and a method for diffusing volatile substances, where the user perceives the fragrance from a short period of time after switching on the device, due to the low thermal inertia of the heater.

### BACKGROUND TO THE INVENTION

Known Device for diffusing volatile substances comprise a wick which is impregnated with the volatile substances to be evaporated and heated so that the desired evaporation takes place.

Usually, the wick is heated by ceramic heaters, which have the disadvantage that their thermal efficiency is not optimized, in particular with regard to rapid temperature changes.

In particular, today's diffuser devices usually operate in two stages. The first is the acceleration phase, until the heater reaches the working temperature. This stage lasts for a few minutes, e.g. 40 minutes. During this stage, the evaporation rate of the fragrance increases progressively with the temperature.

The second stage is stationary, i.e. the temperature remains the same and the evaporation rate of the fragrance is constant.

When the appliance is switched off, there is a cooling period, which is similar to the heating period, and during this period, the fragrances are progressively diffused until they stop evaporating.

With the current situation, the user cannot perceive the fragrance for a long period of time after switching on the device, due to the high thermal inertia of current heaters.

### DESCRIPTION OF THE INVENTION

Therefore, an objective of the present invention is to provide a device and a method for diffusing volatile substances, with which the user perceives the fragrance from a short period of time after switching on the device, due to the low thermal inertia of the heater.

Furthermore, with the device and method for diffusing volatile substances according to the present invention it is possible to create a complete adjustable temperature profile by heating at different temperatures the fragrance to have different evaporation values.

With the device and the method for diffusing volatile substances of the invention, the aforementioned disadvantages are solved, presenting other advantages that will be described below.

The device and method for diffusing volatile substances according to the present invention are described in the respective independent claims, and the dependent claims include additional features that are optional.

In particular, the device for diffusing volatile substances comprises:
a container containing a liquid with volatile substances,
a wick partially housed in the container, which is impregnated with the liquid with volatile substances contained in the container; and
a heater, which heats the wick to promote the diffusion of the volatile substances, wherein the heater comprises a plurality of heating elements arranged around the wick.

In addition, the heater also advantageously comprises a support plate arranged around the wick, on which the heating elements are mounted.

According to a preferred embodiment, the support plate is annular in shape, so that the wick passes through its central hole.

Preferably, the heating elements are arranged equidistant from each other defining a circumference, the wick being arranged at the centre of this circumference.

If desired, the support plate may comprise at least one through hole and one or more notches.

According to a preferred embodiment, the support plate is a printed circuit board and the heating elements are resistors. The resistors may be connected in series and/or in parallel.

In addition, the heater is located around a part of the wick that is located outside the container.

The method for diffusing volatile substances comprises the following steps:
a) heating a wick by means of heating elements of a heater to a temperature between 100° C. and 125° C., e.g. 115° C., in a period of time of less than 10 minutes, e.g. 4 to 5 minutes;
b) cooling the wick to a working temperature between 80° C. and 95° C., e.g. 90° C., within a period of time between 5 and 10 minutes, e.g. 5 minutes;
(c) keeping the wick heated to the working temperature for a period of time between 30 and 45 minutes, e.g. 35 minutes;
d) cooling the wick to a non-evaporation temperature between 20° C. and 40° C., e.g. 30° C., within a period of time of less than 10 minutes, e.g. 6 minutes
e) maintaining the wick at the non-evaporation temperature for a period of time between 10 and 20 minutes, e.g., 15 minutes; and
   restart the diffusion method with step a).

### BRIEF DESCRIPTION OF THE DRAWINGS

For a better understanding of what has been explained above, some drawings are included in which, schematically and only by way of a non-limiting example, a practical case of embodiment is shown.

FIG. 1 is a perspective section view of the Device for diffusing volatile substances according to the present invention;

FIGS. 2a to 2e are perspective views of possible embodiments of the heater of the device for diffusing volatile substances in accordance with the present invention; and

FIG. 3 is a graph showing the temperature changes of the heater over time using the method for diffusing volatile substances according to the present invention.

### DESCRIPTION OF A PREFERRED EMBODIMENT

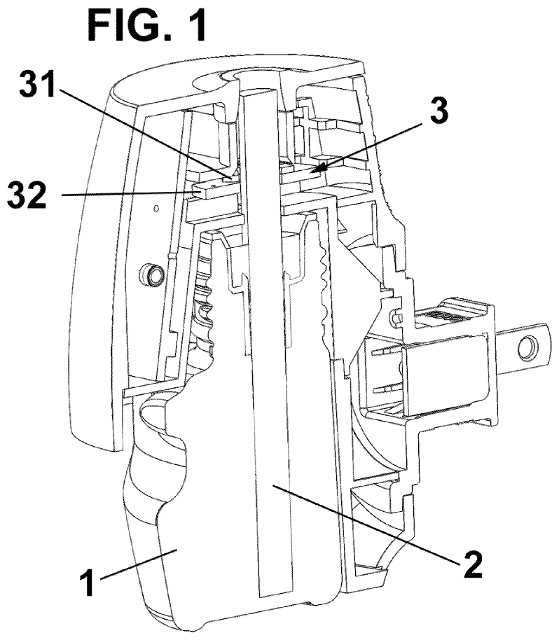

FIG. 1 shows the device for diffusing volatile substances according to the present invention, comprising a container (1) containing a liquid with volatile substances, a wick (2) partially housed in the container (1), which is impregnated with the liquid with volatile substances contained in the container (1), and a heater (3), which heats the wick (2) to promote the diffusion of the volatile substances.

Said heater (3) comprises a plurality of heating elements (31), i.e. two or more heating elements (31), arranged around the wick (2), and a support plate (32) arranged around the wick (2), on which the heating elements (32) are mounted.

Although it could have any suitable shape, the support plate (32) according to the embodiment shown has an annular shape, so that the wick (2) passes through its inner hole.

This support plate (32) is located at a height of the wick (2) above the container (1) according to FIG. 1, i.e. on a part of the wick (2) outside the container (1).

According to a preferred embodiment, the support plate (32) is a printed circuit board, and the heating elements (31) are resistors, which may be connected in series and/or in parallel. In particular, the resistors may be connected all in parallel, or all in series, or connected in pairs in series and in parallel.

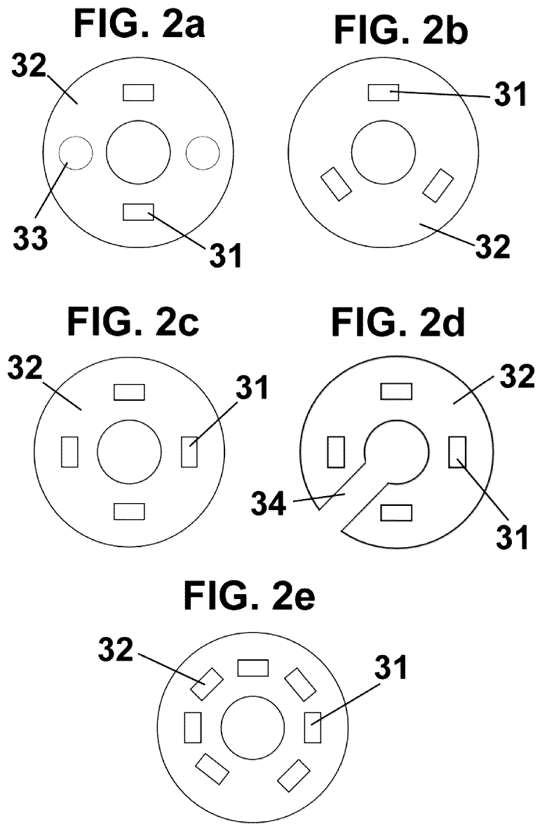

FIGS. 2a to 2e show possible arrangements of the heating elements (31) on the support plate (32) equidistant from each other defining a circumference, the wick (2) being arranged in the centre of this circumference.

According to these provisions, the heating elements (31) arranged on the support plate (32) may be two, three, four, or more.

Furthermore, the support plate (32) may comprise at least one through hole (33) and/or one or more notches (34), to improve the air flow between the wick (2), the support plate (32) and the heating elements (31), improving the evaporation ratio of the volatiles.

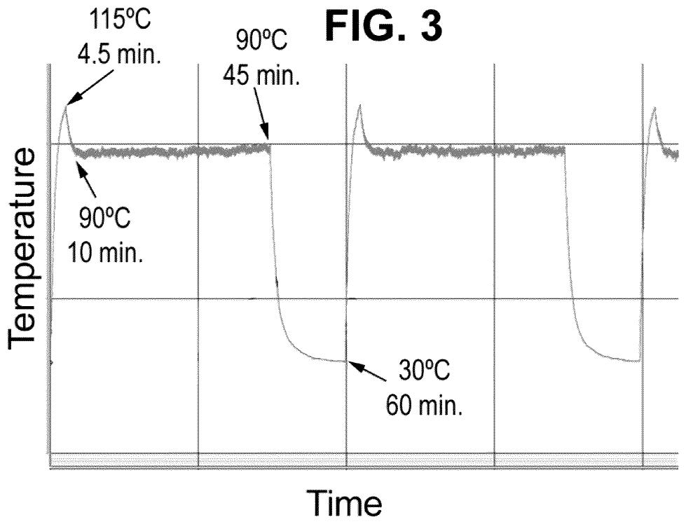

To show the method of diffusion of volatile substances according to the present invention a mode of operation is described with the aid of the graph in FIG. 3, which shows the heating temperature over time achieved by the heating elements (31) of the device according to the present invention.

According to FIG. 3, the operating principle of the device comprises an initial ramp up to the desired peak temperature, e.g. 115° C. in about 4 to 5 minutes, a cooling down to the working temperature, e.g. 90° C., maintained for a period of e.g. about 45 minutes. The next stage is the cooling down to a temperature low enough not to evaporate the volatile substances, e.g. 30° C. After holding at the lower temperature for e.g. about 15 minutes, the cycle starts again with the initial temperature peak.

Although reference has been made to a specific embodiment of the invention, it is obvious to a person skilled in the art that the device for diffusing volatile substances described is susceptible to numerous variations and modifications, and that all the details mentioned can be replaced by technically equivalent ones, without departing from the scope of protection defined by the appended claims.

1. Device for diffusing volatile substances, comprising:
a container containing a liquid with volatile substances,
a wick partially housed in the container, which is impregnated with the liquid with volatile substances contained in the container; and
a heater, which heats the wick to promote the diffusion of the volatile substances, characterized in the heater comprises a plurality of heating elements arranged around the wick.

2. Device for diffusing volatile substances according to claim 1, wherein the heater also comprises a support plate arranged around the wick, on which the heating elements are mounted.

3. Device for diffusing volatile substances according to claim 2, wherein the support plate has an annular shape.

4. Device for diffusing volatile substances according to claim 2, wherein the heating elements are arranged equidistant from each other defining a circumference, the wick being arranged in the center of said circumference.

5. Device for diffusing volatile substances according to claim 2, wherein the support plate has at least one through hole.

6. Device for diffusing volatile substances according to claim 2, wherein the support plate comprises one or more notches.

7. Device for diffusing volatile substances according to claim 2, wherein the support plate is a printed circuit board.

8. Device for diffusing volatile substances according to claim 1, wherein the heating elements are resistors.

9. Device for diffusing volatile substances according to claim 8, wherein the resistors are connected in series and/or in parallel.

10. A method for diffusing volatile substances, characterized in that it comprises the following steps:
a) heating a wick by means of heating elements of a heater to a temperature between 100° C. and 125° C. in a period of time of less than 10 minutes;
(b) cooling the wick to a working temperature of between 80° C. and 95° C. in a period of less than 10 minutes;
(c) keeping the wick heated to the working temperature for a period of time between 30 and 45 minutes;
(d) cooling the wick to an evaporation temperature between 20° C. and 40° C.;
(e) maintaining the wick at the non-vaporising temperature for a period of less than 20 minutes; and
restarting the diffusion method with step a).
